(19) United States
(12) Patent Application Publication
(10) Pub. No.: US 2002/0014430 A1
(43) Pub. Date: Feb. 7, 2002

(54) Needle magazine

(76) Inventor: Lars Morch Groth, Fredensborg (DK)

Correspondence Address: PATENT DEPARTMENT, SKADDEN, ARPS, SLATE, MEAGHER & FLOM LLP, FOUR TIMES SQUARE, NEW YORK, NY, 10036, US

(21) Appl. No.: 09/921,428

(22) Filed: Aug. 2, 2001

Related U.S. Application Data
(60) Provisional application No. 60/225,199, filed on Aug. 14, 2000.

(30) Foreign Application Priority Data
Aug. 3, 2000 (DK) PA 2000 01166

(51) Int. Cl. A61L 15/00

(52) U.S. Cl. 206/438

(57) ABSTRACT

A needle magazine for storing a plurality of needle assemblies and for selectively dispensing the needle assemblies there from. The needle magazine comprises a base member with a number of individual compartments each containing a separate housing. Each compartment is at the upper surface provided with a movable lid, which can be opened to gain access to the interior of each individual compartment. Each housing contains one needle assembly, and is mounted in the compartments of the base member in a way making it possible to rotate the housing and thereby the needle assembly without rotating the base member, by providing each housing with a circumferential flange, which is fitted into a circumferential track on the interior surface of each compartment of the base member.

The invention relates to a needle magazine for storing, dispensing and collecting a number of needle assemblies.

Medical injection devices are used to deliver selected doses of medication to patients. Some medication, such as insulin is self-administered. The typical diabetes patient will require injections of insulin several times during the course of the day. In order to prevent infections it is recommended to use a sterile needle assembly for each new injection. Needle assemblies are often delivered in separate housing where each housing contains only one sterile needle assembly. Such a housing is described in U.S. Pat. No. 5,971,966. Using a needle assembly of this kind requires the patient to open the housing and to fasten the needle assembly onto the injection device prior to each injection. Usually the needle hub of the needle assembly is provided with an internal thread and force fitted into the housing. A typical injection device is on the tip provided with an external thread, which fits in the internal thread of the needle hub. In order to connect the needle assembly and the injection device the two elements most be rotated relatively to each other. In order to facilitate a better grip on the needle assembly, this is usually kept in the housing when mounting it on the injection device. The storage of sterile needle assemblies of this type and the final disposal of used needle assemblies present a problem, since new sterile needle assemblies are often carried loosely in purses or briefcases, and used needle assemblies are often disposed of unsafely.

To overcome these problems a needle magazine for storage and dispensing a plurality of needle assemblies has been developed. An example of such prior art magazine is shown in WO 92.12743, and comprises a plurality of housings connected together on a strip and guided in a magazine. The needle assemblies located in the housings are used one by one, and returned to the housing and disposed of after use.

Another example of a prior art needle magazine is known form EP 990.446. This needle magazine comprises of a base member having a plurality of compartments each compartment containing a housing. Each housing contains a needle assembly, and can be accessed through the upper surface. The housings are rotational locked to the base member due to a series of anti-rotation ribs located at the distal end of the housing. Each housing has an internal thread facilitating the correct mounting of the needle assemblies onto the injection device.

For safety reasons needle assemblies most be collected after use and disposed of in a safe manner. A needle magazine providing adequate safety not only has to be able to store and selectively dispense a plurality of needle assemblies, but also to collect the used needle assemblies, such that the entire needle magazine can be disposed of in a safe manner.

For self-administering insulin there are primarily two types of injection devices available today, both types having an external thread provided on the tip of the injection device for connecting with the internal thread of a needle assembly. One type of injection devices being a pen shaped injection device, which is symmetrical around the axis of rotation used when mounting the needle assembly. The other type is a compact injector, or compact doser, which is an injection device with a non-symmetrical design. Since these so-called compact injectors is not symmetrical around the axis of rotation used when mounting the needle assembly, an ordinary needle magazine, as the one known from EP 990.446 cannot accommodate such an injection device.

It is an object of the present invention to provide a needle magazine for storing, dispensing and collecting a plurality of needle assemblies by which a great number of needle assemblies can be contained in the needle magazine and which magazine overcomes the inconveniences of the prior art needle magazines.

It is further an object of the present invention to provide a needle magazine for storing, dispensing and collecting a plurality of needle assemblies, and by which there are no or only limited risk of accidental needle-stick injuries.

Finally it is the object of the present invention to provide a needle magazine where the needle assemblies can be positioned onto the injection device and rotated individually and independently of the remaining part of the needle magazine, thereby making it possible to mount the needle assemblies onto a non symmetrically compact injector and to dismount the needle assemblies from such a injection device without having to rotate neither the injection device nor the needle magazine.

This is obtained by a needle magazine for storing a plurality of needle assemblies and for selectively dispensing said needle assemblies there from, comprising: a plurality of housings, each housing containing one of said needle assemblies, and a base member including a number of compartments, each compartment containing one of said housings, said base member having a upper surface and a bottom surface, through which upper surface each housing can be accessed, whereby access to one of said needle assemblies located in one of said housings can be provided,

which needle magazine according to the invention is characterized in that each housing extends beyond said bottom surface, and that each housing can be separately rotated inside said compartment in order to mount said needle assembly onto a fluid transfer apparatus

The needle assemblies in the needle magazine are confined in separate, sterile housings, which housings are individually and loosely hung in the compartments of the needle magazine. In this way each housing can be individually rotated relatively to the needle magazine, and since each needle assembly are rotational connected to the housing, rotation of the housing causes the needle assembly to rotate. When connecting the needle assembly to the injection device, the need for rotating the injection device is now eliminated, since each housing can rotate freely.

When, as disclosed in claim 2, the needle assemblies comprises a needle cannula fasten to a needle hub, which needle hub is force fitted into said housing, it is ensured that each needle assembly rotates together with the housing when the housing is rotated.

When, as disclosed in claim 3, each housing is secured in each compartment by having an external flange on each housing fitted in a circular sleeve provided on the interior surface of each compartment, it is, in a very convenient way, ensured that the housing can rotate relatively to the base member of the needle magazine.

When, as disclosed in claim 4, the housing, at least on the bottom part, is provided with a roughed surface, it is ensured that the user is provided with a solid grip when rotating said housing.

When, as disclosed in claim 5, each housing is sealed by a pealable label, it is ensured that the interior of each housing is kept sterile.

When, as disclosed in claim 6, each compartment at the upper surface is provided with a movable lid, it is ensured that the user in an easy way can gain access to each housing and to each needle assembly.

When, as disclosed in claim 7, the movable lid is provided with a childproof closure, it is ensured that children cannot gain access to the needle assemblies located in the magazine. The childproof closure comprises, as disclosed in claim 8, a hook-shaped element provided on said lid, a protrusion provided on a lever connected to said base member and an additional protrusion located on said lid, said hook-shaped member encompassing said protrusion and being disconnected there from by pressing said lever towards said base member whereby said lid can be lifted overcoming the engagement of said additional protrusion in said base member.

When, as disclosed in claim 9, the base member has a number of weakened lines along which lines said base member could be divided into a random number of smaller base members, it is ensured that the user can customize the needle magazine by breaking it up into a random number of smaller needle magazines only containing the number of needle assemblies required by the specific user.

The invention will be explained more fully below in connection with a preferred embodiment and with reference to the drawings in which:

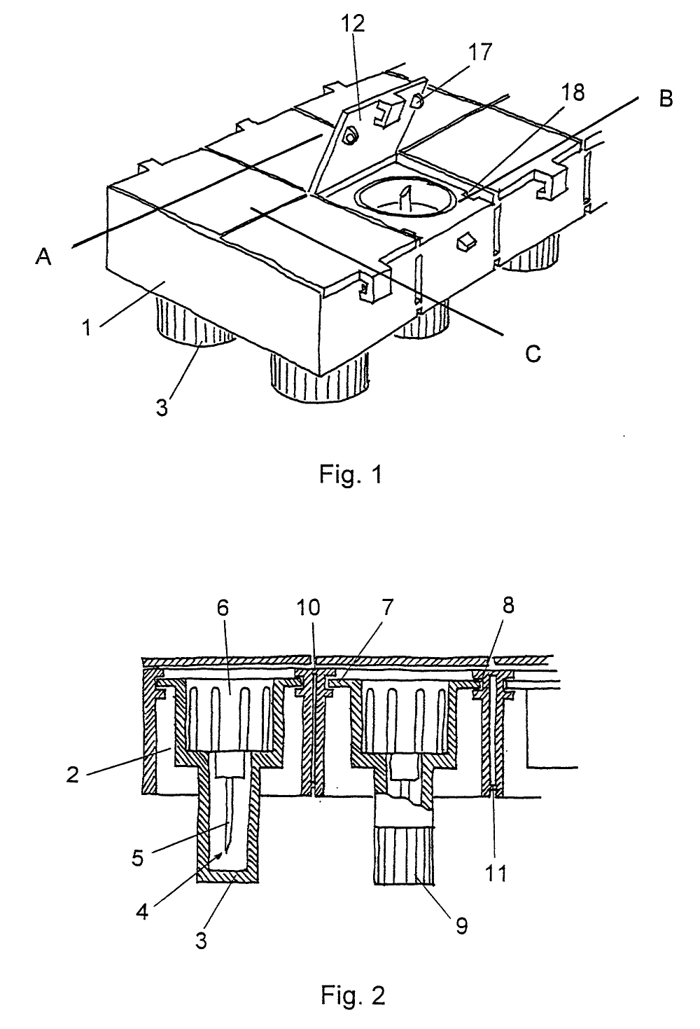

FIG. 1 Shows a perspective view of the needle magazine according to the invention.

FIG. 2 Shows a view along line A in FIG. 1.

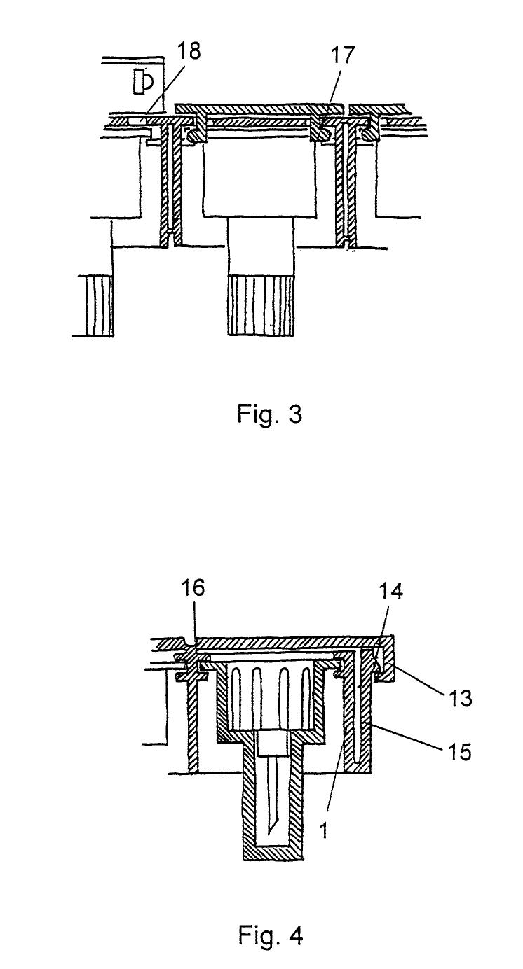

FIG. 3 Shows a view along line B in FIG. 1.

FIG. 4 Shows a view along line C in FIG. 1.

The figures are schematic and simplified for clarity, and they just show details, which are essential to the understanding of the invention, while other details are left out. Throughout, the same reference numerals are used for identical or corresponding parts.

FIGS. 1 to 4 shows the needle magazine according to the invention. The needle magazine comprises a base member 1 with a number of individual compartments 2, which compartments 2 each contains a housing 3. Each compartment 2 is at the upper surface provided with a movable lid 6, which can be opened to gain access to the interior of each individual compartment 2.

Each housing 3 again contains one needle assembly 4, comprising a needle cannula 5 and a needle hub 6. A not shown pealable label seals each housing 3 such that each needle assembly 4 can be maintained sterile as long as it is stored in the housing 3. The label may e.g. be made from paper, which does not allow germs to pass but is permeable to hot steam used to sterilize the needle assembly 4 in the housing 3.

Each housing 3 containing one needle assembly 4 is mounted in the base member 1 in a way making it possible to rotate the housing 3 and the needle assembly 4 without rotating the base member 1. This is done by providing each housing 3 with a circumferential flange 7, which is fitted into a circumferential track 8 on the interior surface of each compartment 2 of the base member 1. Each needle assembly 4 is rotational locked to each housing, such that the needle assembly 4 rotates together with the housing 3 once the housing 3 is rotated. This rotational connection is preferably made by initially force fitting the needle hub 6 into the housing 3, or by providing the needle hub 6 with longitudinal tracks or protrusions fitting into similar tracks or protrusions located on the interior surface of the housing 3. To facilitate the user in rotating the housing 3, the distal end of each housing 3 can be provided with a roughed surface 9.

The housing 3 can be separated from the housing by disengaging the flange 7 from the track 8. This is possible due to both the base member 1 and the housing 3 being made from a polymeric material, which materials are usually bendable. In this way used needle assemblies can easily be replaced with new sterile needle assemblies 4.

The base member 1 is, as shown, divided into a number of individual compartments 2. These compartments 2 are made integral with each other in the transversal direction and hinged to each other in the longitudinal direction by hinges 10, 11. The upper hinges 10 is typical a film hinges which extends all the way in the transversal direction, while the lower hinges 11 is a single-point hinges only connecting the compartments 2 at a single point. By breaking the base member 1 along the hinges 10, 11, a number of minor base members containing only the number of compartments 2 integral connected in the transversal direction is provided. As shown in FIG. 1 the number of compartments 2 integral connected in the transversal direction is typically two due to the location of the locking mechanism of the lid 12, while the number of longitudinal hinged compartments 2 can be endless. When breaking up the base member 1 into minor base members the user decides how large these minor base member should be, however a typical user often needs such minor base member to contain two or four needle assemblies.

As pointed out earlier there are primarily two types of injection devices available today for injecting insulin, both types has an external thread provided on the tip of the injection device for connecting with the internal thread of a needle assembly. One type of injection devices being a pen shaped injection device, which is symmetrical around the axis of rotation used when mounting the needle assembly. The other type is a compact injector, which is an injection device with a non-symmetrical design.

When connecting a pen shaped injection device with a needle assembly 4 located in the needle magazine according to the invention described herein, the user first places the needle magazine on a vertical surface such as a table, and opens the lid 12. Then the external thread of the injection device is moved forward into the internal thread of the needle hub 6, and the injection device is rotated relatively to the needle magazine. After use the needle assembly 4 is relocated in the needle magazine by moving the needle assembly 4 now carried on the injection device into the housing and rotating the injection device, once the needle assembly is disconnected from the injection device this can be removed and the lid closed.

When connecting a compact injector with a needle assembly 4 located in the needle magazine according to the invention described herein, the user first places the needle magazine standing horizontally on a vertical surface such as a table, and opens the lid 12. The external thread of the compact injector is then moved forward into the internal thread of the needle hub 6, and the housing 3 carrying the needle assembly 4 is rotated utilizing the roughed surface 9 on the distal end of the housing 3. After use the needle assembly 4 is relocated in the needle magazine by moving the needle assembly 4 now carried on the compact injector into the housing 3 and rotating the housing 3 in the opposite direction. Once the needle assembly 4 is disconnected from the compact injector this can be removed and the lid 12 closed.

The lid 12 is, as best seen in FIG. 4, provided with a locking mechanism comprising a hook-shaped element 13 located on the lid 12, and a protrusion 14 provided on the base member 1. The part of the base member 1 carrying the protrusion 14 is made as a lever 15 connected to the base part at a distance from the protrusion 14. In order to open the lid 12 the user simply has to press the lever 15 towards the base member with one hand while the hook-shaped element 13 then automatically slips over the protrusion 14. In order to provide a more childproof closure a number of additional protrusions 17 can be provided on the lid. These additional protrusions engage an opening 18 in the base member 1 in such a way, that a person, in order to open the lid 12, both has to press the lever 15 towards the base member 1 and to pull the lid 1 upwardly at the same time. This movement is especially difficult to be performed by small children; therefore gaining access to the interior of the compartment is almost impossible for small children.

Some preferred embodiments have been shown in the foregoing, but it should be stressed that the invention is not limited to these, but may be embodied in other ways within the subject matter defined in the following claims.

1. A needle magazine for storing a plurality of needle assemblies and for selectively dispensing said needle assemblies there from, comprising:
a plurality of housings, each housing containing one of said needle assemblies, and
a base member including a number of compartments, each compartment containing one of said housings, said base member having a upper surface and a bottom surface, through which upper surface each housing can be accessed, whereby access to one of said needle assemblies located in one of said housings can be provided,
Characterized in that
each housing extends beyond said bottom surface, and
that each housing can be separately rotated inside said compartment in order to mount said needle assembly onto a fluid transfer apparatus

2. A needle magazine for storing a plurality of needle assemblies according to claim 1, characterized in that said needle assemblies comprises a needle cannula fasten to a needle hub, which needle hub is force fitted into said housing.

3. A needle magazine for storing a plurality of needle assemblies according to claim 1 or 2, characterized in that each housing is secured in each compartment by having an external flange on each housing fitted in a circular sleeve provided on the interior surface of each compartment.

4. A needle magazine for storing a plurality of needle assemblies according to anyone of the claims 1 to 3, characterized in that said housing at least on the bottom part is provided with a roughed surface in order to provide the user with a better grip when rotating said housing.

5. A needle magazine for storing a plurality of needle assemblies according to anyone of the claims 1 to 4, characterized in that each housing is sealed by a pealable label.

6. A needle magazine for storing a plurality of needle assemblies according anyone of the claims 1 to 5, characterized in that each compartment at the upper surface is provided with a movable lid.

7. A needle magazine for storing a plurality of needle assemblies according to claim 6, characterized in that said movable lid is provided with a childproof closure

8. A needle magazine for storing a plurality of needle assemblies according to claim 7, characterized in that said childproof closure comprises a hook-shaped element provided on said lid, a protrusion provided on a lever connected to said base member and an additional protrusion located on said lid, said hook-shaped member encompassing said protrusion and being disconnected there from by pressing said lever towards said base member whereby said lid can be lifted overcoming the engagement of said additional protrusion in said base member.

9. A needle magazine for storing a plurality of needle assemblies according anyone of the preceding claims, characterized in that said base member has a number of weakened lines along which lines said base member can be divided into a random number of smaller base members.
